(19) United States
(12) Patent Application Publication
(10) Pub. No.: US 2013/0272961 A1
(43) Pub. Date: Oct. 17, 2013

(54) RADIOIODINATED GUANIDINES

(71) Applicants: Michelle E. Avory, Wendover (GB); Robert James Domett Nairne, Amersham (GB); Harry Jon Wadsworth, Bishops Stortford (GB)

(72) Inventors: Michelle E. Avory, Wendover (GB); Robert James Domett Nairne, Amersham (GB); Harry Jon Wadsworth, Bishops Stortford (GB)

(73) Assignee: GE HEALTHCARE LIMITED, Buckinghamshire (GB)

(21) Appl. No.: 13/995,528

(22) PCT Filed: Dec. 20, 2011
(86) PCT No.: PCT/EP11/73380

§ 371 Date: Jun. 19, 2013

Related U.S. Application Data
(60) Provisional application No. 61/424,719, filed on Dec. 20, 2010.

(30) Foreign Application Priority Data
Dec. 20, 2010 (GB) 1021517.6

(51) Int. Cl. A61K 51/04

(52) U.S. Cl. CPC A61K 51/0453
USPC 424/1.85; 548/255; 548/247

(57) ABSTRACT

The present invention provides novel radioiodinated guanidines. Also provided are methods of preparation of said radioiodinated guanidines from non-radioactive precursors, as well as radiopharmaceutical compositions comprising such radioiodinated guanidines. The invention also provides in vivo imaging methods using the radioiodinated guanidines.

### FIELD OF THE INVENTION

The present invention provides novel radioiodinated guanidines. Also provided are methods of preparation of said radioiodinated guanidines from non-radioactive precursors, as well as radiopharmaceutical compositions comprising such radioiodinated guanidines. The invention also provides in vivo imaging methods using the radioiodinated guanidines.

### BACKGROUND TO THE INVENTION

Meta-iodobenzylguanidine (mIBG) is an analogue of the neurotransmitter norepinephrine with affinity for the sympathetic nervous system and related tumours. Radioiodinated mIBG labelled with ¹²³I is used as a radiopharmaceutical for in vivo imaging to assist in the diagnosis of various pathophysiological conditions of the heart, as well as neuroendocrine tumours, whereas ¹³¹I-mIBG is used for therapy of neuroblastoma and pheochromocytoma. For a review, see Vaidyanathan [Quart. J. Nucl. Med. Mol. Imaging, 52, 351-368 (2008)].

The applications of “click chemistry” in biomedical research, including radiochemistry, have been reviewed by Nwe et al [Cancer Biother. Radiopharm., 24(3), 289-302 (2009)]. As noted therein, the main interest has been in the PET radioisotope ¹⁸F (and to a lesser extent ¹¹C), plus “click to chelate” approaches for radiometals suitable for SPECT imaging such as ^{99m}Tc or ¹¹¹In. ¹⁸F click-labelling of targeting peptides, giving products incorporating an ¹⁸F-fluoroalkyl-substituted triazole have been reported by Li et al [Bioconj. Chem., 18(6), 1987-1994 (2007)], and Hausner et al [J. Med. Chem., 51(19), 5901-5904 (2008)].

WO 2006/067376 discloses a method for labelling a vector comprising reaction of a compound of formula (I) with a compound of formula (II):

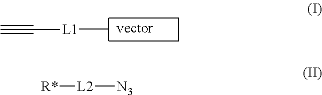

or,
a compound of formula (III) with a compound of formula (IV)

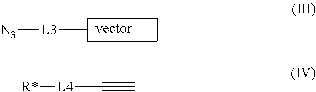

in the presence of a Cu(I) catalyst, wherein:
L1, L2, L3, and L4 are each Linker groups;
R* is a reporter moiety which comprises a radionuclide;
   to give a conjugate of formula (V) or (VI) respectively:

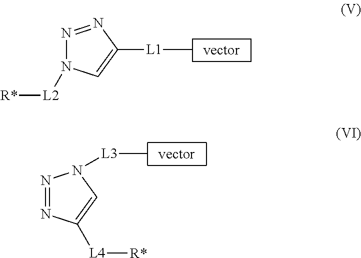

wherein L1, L2, L3, L4, and R* are as defined above.

R* of WO 2006/067376 is a reporter moiety which comprises a radionuclide for example a positron-emitting radionuclide. Suitable positron-emitting radionuclides for this purpose are said to include ¹¹C, ¹⁸F, ⁷⁵Br, ⁷⁶Br, ¹²⁴I, ⁸²Rb, ⁶⁸Ga, ⁶⁴Cu and ⁶²Cu, of which ¹¹C and ¹⁸F are preferred. Other useful radionuclides are stated to include ¹²³I, ¹²⁵I, ¹³¹I, ²¹¹At, ^{99m}Tc, and ¹¹¹In.

WO 2007/148089 discloses a method for radiolabelling a vector comprising reaction of a compound of formula (I) with a compound of formula (II):

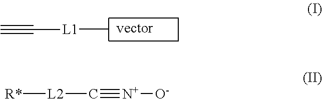

or, a compound of formula (III) with a compound of formula (IV):

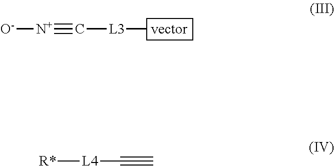

in the presence of a Cu(I) catalyst, wherein:
L1, L2, L3, and L4 are each Linker groups;
R* is a reporter moiety which comprises a radionuclide;
   to give a conjugate of formula (V) or (VI) respectively:

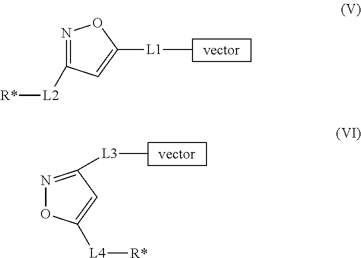

In both WO 2006/067376 and WO 2007/148089, metallic radionuclides are stated to be suitably incorporated into a chelating agent, for example by direct incorporation by methods known to the person skilled in the art.

WO 2006/116629 (Siemens Medical Solutions USA, Inc.) discloses a method of preparation of a radiolabelled ligand or substrate having affinity for a target biomacromolecule, the method comprising:
(a) reacting a first compound comprising
   (i) a first molecular structure;
   (ii) a leaving group;
   (iii) a first functional group capable of participating in a click chemistry reaction; and optionally,
   (iv) a linker between the first functional group and the molecular structure, with a radioactive reagent under conditions sufficient to displace the leaving group with a radioactive component of the radioactive reagent to form a first radioactive compound;
(b) providing a second compound comprising
   (i) a second molecular structure;
   (ii) a second complementary functional group capable of participating in a click chemistry reaction with the first functional group, wherein the second compound optionally comprises a linker between the second compound and the second functional group;
(c) reacting the first functional group of the first radioactive compound with the complementary functional group of the second compound via a click chemistry reaction to form the radioactive ligand or substrate; and
(d) isolating the radioactive ligand or substrate.

WO 2006/116629 teaches that the method therein is suitable for use with the radioisotopes: ¹²⁴I, ¹⁸F, ¹¹C, ¹³N and ¹⁵O with preferred radioisotopes being: ¹⁸F, ¹¹C, ¹²³I, ¹²⁴I, ¹²⁷I, ¹³¹I, ⁷⁶Br, ⁶⁴Cu, ^{99m}Tc, ⁹⁰Y, ⁶⁷Ga, ⁵¹Cr, ¹⁹²Ir, ⁹⁹Mo, ¹⁵³Sm and ²⁰¹Tl. WO 2006/116629 teaches that other radioisotopes that may be employed include: ⁷²As, ⁷⁴As, ⁷⁵Br, ⁵⁵Co, ⁶¹Cu, ⁶⁷Cu, ⁶⁸Ga, ⁶⁸Ge, ¹²⁵I, ¹³²I, ¹¹¹In, ⁵²Mn, ²⁰³Pb and ⁹⁷Ru. WO 2006/116629 does not, however, provide any specific teaching on how to apply the method to the radioiodination of biological molecules.

Radioiodinated mIBG derivatives are, however, known to suffer from metabolic deiodination in vivo, which is more pronounced for no-carrier-added preparations [Faraahati et al, J. Nucl. Med., 38, 447-451 (1997)]. For radioiodine-containing radiopharmaceuticals, the impact of such deiodination is typically unwanted radioiodide uptake in the thyroid, with consequent risk of radiation dose to the thyroid. Such thyroid uptake can be suppressed by co-administration of excess non-radioactive iodide ion to the patient together with the radiopharmaceutical, so the risk of radiation does to the thyroid is minimised. From an imaging perspective, however, it is always undesirable to have such in vivo deiodination, since some signal from the desired agent is lost, and potentially competing or background signal from the radioiodine-containing metabolites (typically radioiodide) is generated. There is therefore a need for radioiodinated mIBG analogues which are resistant to in vivo deiodination.

### THE PRESENT INVENTION

The present invention provides radioiodinated guanidine analogues comprising triazole or isoxazole rings. The triazole and isoxazole rings do not hydrolyse and are highly stable to oxidation and reduction, meaning that the labelled guanidine has high in vivo stability. The triazole ring is also comparable to an amide in size and polarity. The triazole and isoxazole rings of the guanidines of Formula (I) of the present invention are not expected to be recognized by thyroid deiodination enzymes known to metabolise iodo-tyrosine and iodo-benzene species, and are thus expected to be sufficiently stable in vivo for radiopharmaceutical imaging and/or radiotherapy.

The present radioiodinated guanidines can be synthesised readily using either click chemistry, or organometallic precursors.

### DETAILED DESCRIPTION OF THE INVENTION

In a first aspect, the present invention provides radioiodinated guanidine of Formula (I):

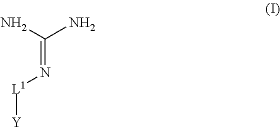

where:
Y is a Y¹ or Y² group:

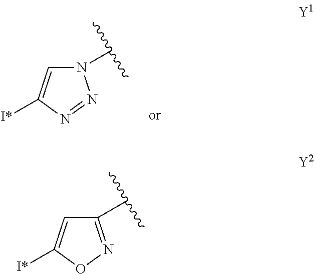

L¹ is a linker group of formula -(A)ₙ- where n is an integer of value 1 to 4, and each A group is independently chosen from —CH₂— and —C₆H₄—;

I* is a radioisotope of iodine.

The term “radioiodinated” has its conventional meaning, i.e. a radiolabelled compound wherein the radioisotope used for the radiolabelling is a radioisotope of iodine. The term “radioisotope of iodine” has its conventional meaning, i.e. an isotope of the element iodine that is radioactive. Suitable such radioisotopes include: ¹²³I, ¹²⁴I, ¹²⁵I and ¹³¹I.

The term “guanidine” has its conventional meaning, i.e. a compound of formula HN═C(NH₂)₂, also sometimes termed an imido-urea or amidocarbonic acid.

Preferred Aspects.

Preferred radioisotopes of iodine for use in the present invention are those suitable for medical imaging in vivo using PET or SPECT, preferably ¹²³I, ¹²⁴I or ¹³¹I, more preferably ¹²³I or ¹²⁴I, most preferably ¹²³I.

A preferred radioiodinated guanidine of the first aspect is where Y is Y¹, i.e. the radioiodine isotope is attached to a triazole ring.

In Formula (I), n is preferably 1 to 3, more preferably 1 or 2, most preferably 1. In Formula (I), L¹ is preferably —(CH₂)ₙ—, more preferably —(CH₂)ₙ— with the preferred values of n.

The radioiodinated guanidines of Formula (I) may be obtained as described in the second or third aspects (below). The preparation method of the second aspect (via Precursor IA) is preferred, since that comprises only a single step in which radioactive manipulations are involved.

Included within the scope of the first aspect is an imaging agent which comprises the radioiodinated guanidine of Formula (I). By the term “imaging agent” is meant a compound suitable for imaging the mammalian body. Preferably, the mammal is an intact mammalian body in vivo, and is more preferably a human subject. Preferably, the imaging agent can be administered to the mammalian body in a minimally invasive manner, i.e. without a substantial health risk to the mammalian subject when carried out under professional medical expertise. Such minimally invasive administration is preferably intravenous administration into a peripheral vein of said subject, without the need for local or general anaesthetic. The imaging agents of the first aspect are preferably used as radiopharmaceutical compositions, as described in the fourth aspect (below).

In a second aspect, the present invention provides a method of preparation of the radioiodinated guanidine of Formula (I) as defined in the first aspect, where said method comprises:
(i) provision of a precursor of Formula (IA)

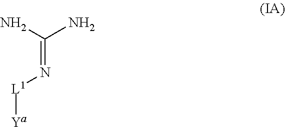

where:
L¹ is as defined in the first aspect;
Y^{a} is a Y^{1a} or Y^{2a} group:

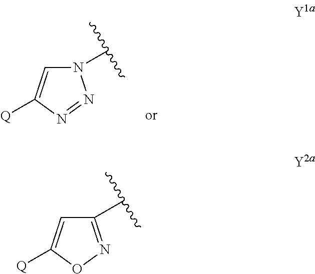

wherein Q is R^{a}₃Sn— or KF₃B—, where each R^{a} is independently C₁₋₄ alkyl;
(ii) reaction of said precursor with radioactive iodide ion in the presence of an oxidising agent to give the radioiodinated guanidine of Formula (I).

Preferred embodiments of L¹, n and the radioactive isotope of iodine in the second aspect are as defined in the first aspect. The precursor of Formula (IA) is suitably non-radioactive, so can be prepared and purified by conventional means without the need for radiation handling safety precautions.

By the term “oxidising agent” is meant an oxidant capable of oxidising iodide ion to form the electrophilic species (HOI, H₂OI), wherein the active iodinating agent is I⁺. Suitable oxidising agents are described by Bolton [J. Lab. Comp. Radiopharm., 45, 485-528 (2002)], and Eersels et al [J. Lab. Comp. Radiopharm., 48, 241-257 (2005)] and include peracetic acid and N-chloro compounds, such as chloramine-T, iodogen, iodogen tubes and succinimides. Preferred oxidising agents are peracetic acid (which is commercially available) at pH ca. 4, and hydrogen peroxide/aqueous HCl at pH ca. 1. Iodogen tubes are commercially available from Thermo Scientific Pierce Protein Research Products.

By the term “radioactive iodide ion” is meant a radioisotope of iodine (I* as defined above), in the chemical form of iodide ion (I⁻).

When Q is R^{a}₃Sn—, the radioiodination method of the second aspect is carried out as described by Bolton [J. Lab. Comp. Radiopharm., 45, 485-528 (2002)] and Eersels et al [J. Lab. Comp. Radiopharm., 48, 241-257 (2005)]. The organotin precursors are prepared as described by Ali et al [Synthesis, 423-445 (1996)].

In Y^{a}, when Q is KF₃B—, that corresponds to a potassium trifluoroborate derivative. When Q is KF₃B—, the radioiodination reaction method of the second aspect can be carried out as described by Kabalka et al [J. Lab. Comp. Radiopharm., 48, 359-362 (2005)], who use peracetic acid as the oxidising agent. Precursors where Q is KF₃B— can be obtained from the corresponding alkyne as described by Kabalka et al [J. Lab. Comp. Radiopharm., 48, 359-362 (2005) and, J. Lab. Comp. Radiopharm., 49, 11-15 (2006)]. The potassium trifluoroborate precursors are stated to be crystalline solids, which are stable to both air and water.

In the second aspect, Q is preferably R^{a}₃Sn. Preferred R^{a}₃Sn— groups are Bu₃Sn— or Me₃Sn—, preferably Me₃Sn—.

The radioiodination reaction of the second aspect may be effected in a suitable solvent, for example acetonitrile, a C₁₋₄ alkylalcohol, dimethylformamide, tetrahydrofuran (THF), or dimethylsulfoxide, or mixtures thereof, or aqueous mixtures thereof, or in water. Aqueous buffers can also be used. The pH will depend on the oxidant used, and will typically be pH 0 to 1 when eg. hydrogen peroxide/aqueous acid is used, or in the range pH 6-8 when iodogen or iodogen tubes are used. The radioiodination reaction temperature is preferably 10 to 60° C., more preferably at 15 to 50° C., most preferably at ambient temperature (typically 15-37° C.). Organic solvents such as acetonitrile or THF and/or the use of more elevated temperature may conveniently be used to solubilise any precursors of Formula (IA) which are poorly soluble in water.

In a third aspect, the present invention provides a method of preparation of the radioiodinated guanidine of Formula (I) as defined in the first aspect, where said method comprises:
(i) provision of a precursor of Formula (IB)

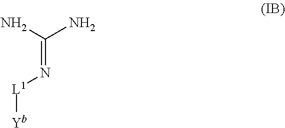

where:
L¹ is as defined in the first aspect;
Y^{b} is a Y^{1b} or Y^{2b} group:

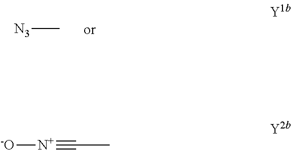

(ii) reaction of said precursor with a compound of Formula (II):

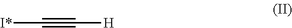

in the presence of a click cycloaddition catalyst, to give the radioiodinated guanidine of Formula (I) via click cycloaddition,
wherein I* is a radioisotope of iodine, as defined in the first aspect.

The precursor of Formula (IB) is suitably non-radioactive, so can be prepared and purified by conventional means without the need for radiation handling safety precautions.

In Formula (IB), Y can be either an azide substituent (Y═Y^{1b}), or an isonitrile oxide substituent Y═Y^{2b}).

By the term “click cycloaddition catalyst” is meant a catalyst known to catalyse the click (alkyne plus azide) or click (alkyne plus isonitrile oxide) cycloaddition reaction of the first aspect. Suitable such catalysts are known in the art for use in click cycloaddition reactions. Preferred such catalysts include Cu(I), and are described below. Further details of suitable catalysts are described by Wu and Fokin [Aldrichim. Acta, 40(1), 7-17 (2007)] and Meldal and Tornoe [Chem. Rev., 108, 2952-3015 (2008)]. The applications of “click chemistry” in biomedical research, including radiochemistry, have been reviewed by Nwe et al [Cancer Biother. Radiopharm., 24(3), 289-302 (2009)].

Preferred Aspects.

A preferred click cycloaddition catalyst comprises Cu(I). The Cu(I) catalyst is present in an amount sufficient for the reaction to progress, typically either in a catalytic amount or in excess, such as 0.02 to 1.5 molar equivalents relative to the compound of Formula (Ia) or (Ib). Suitable Cu(I) catalysts include Cu(I) salts such as CuI or [Cu(NCCH₃)₄][PF₆], but advantageously Cu(II) salts such as copper (II) sulphate may be used in the presence of a reducing agent to generate Cu(I) in situ. Suitable reducing agents include: ascorbic acid or a salt thereof for example sodium ascorbate, hydroquinone, metallic copper, glutathione, cysteine, Fe²⁺, or Co²⁺. Cu(I) is also intrinsically present on the surface of elemental copper particles, thus elemental copper, for example in the form of powder or granules may also be used as catalyst. Elemental copper, with a controlled particle size is a preferred source of the Cu(I) catalyst. A more preferred such catalyst is elemental copper as copper powder, having a particle size in the range 0.001 to 1 mm, preferably 0.1 mm to 0.7 mm, more preferably around 0.4 mm. Alternatively, coiled copper wire can be used with a diameter in the range of 0.01 to 1.0 mm, preferably 0.05 to 0.5 mm, and more preferably with a diameter of 0.1 mm. The Cu(I) catalyst may optionally be used in the presence of bathophenanthroline, which is used to stabilise Cu(I) in click chemistry.

In the method of the third aspect, the compound of Formula (II) may optionally be generated in situ by deprotection of a compound of Formula (IIa):

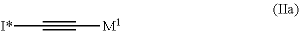

wherein M¹ is an alkyne-protecting group, and I* is as defined for Formula (II). Preferred aspects of I* in Formula (IIa), are as described for Formula (II).

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Suitable alkyne protecting groups are described in Protective Groups in Organic Synthesis, Theodora W. Greene and Peter G. M. Wuts, Chapter 8, pages 927-933, 4^{th} edition (John Wiley & Sons, 2007), and include: an trialkylsilyl group where each alkyl group is independently C₁₋₄ alkyl; an aryldialkylsilyl group where the aryl group is preferably benzyl or biphenyl and the alkyl groups are each independently C₁₋₄ alkyl; hydroxymethyl or 2-(2-hydroxypropyl). A preferred such alkyne protecting group is trimethylsilyl. The protected iodoalkynes of Formula (IIa) have the advantages that the volatility of the radioactive iodoalkyne can be controlled, and that the desired alkyne of Formula (II) can be generated in a controlled manner in situ so that the efficiency of the reaction with the precursor of Formula (IB) is maximised.

The click cycloaddition method of the third aspect may be effected in a suitable solvent, for example acetonitrile, a C₁₋₄ alkylalcohol, dimethylformamide, tetrahydrofuran, or dimethylsulfoxide, or aqueous mixtures of any thereof, or in water. Aqueous buffers can be used in the pH range of 4-8, more preferably 5-7. The reaction temperature is preferably 5 to 100° C., more preferably at 75 to 85° C., most preferably at ambient temperature (typically 15-37° C.). The click cycloaddition may optionally be carried out in the presence of an organic base, as is described by Meldal and Tornoe [Chem. Rev. 108, 2952, Table 1 (2008)].

A preferred precursor of Formula (IB) has Y^{b}═Y^{1b}. One reason is that the isonitrile oxides are typically less stable than azides. Consequently, whilst the azide of Formula (IB, Y^{b}═Y^{1b}) can be isolated and purified, the isonitrile oxide of Formula (IB, Y^{b}═Y^{2b}) will typically need to be generated in situ.

The non-radioactive precursor compound of Formula (IB), where Y^{b} is Y^{1b} (azido derivatives) may be prepared by either:
(i) reaction of the corresponding bromo-guanidine with sodium azide;
(ii) conversion of the corresponding hydroxy-guanidine to a tosylate or mesylate derivative, and subsequent reaction with sodium azide.

The non-radioactive precursor compound of Formula (IB), where Y^{b} is Y^{2b} (isonitrile oxide derivatives) may be prepared by the methods described by Ku et al [Org. Lett., 3(26), 4185-4187 (2001)], and references therein. Thus, they are typically generated in situ by treatment of an alpha-halo aldoxime with an organic base such as triethylamine. A preferred method of generation, as well as conditions for the subsequent click cyclisation to the desired isoxazole are described by Hansen et al [J. Org. Chem., 70(19), 7761-7764 (2005)]. Hansen et al generate the desired alpha-halo aldoxime in situ by reaction of the corresponding aldehyde with chloramine-T trihydrate, and then dechlorinating this with sodium hydroxide. The corresponding aldoxime is prepared by reacting the corresponding aldehyde with hydroxylamine hydrochloride at pH 9-10. See also K. B. G. Torsell “Nitrile Oxides, Nitrones and Nitronates in Organic Synthesis” [VCH, New York (1988)].

Included within the scope of this third aspect, is the option of using an aldoxime precursor, wherein instead of Y^{2b}, Y^{b} is chosen to be (HO)N═CH—, so that the isonitrile oxide (Y^{b}═Y^{2b}) is generated in situ.

The preparation methods of the second and third aspects are preferably carried out in an aseptic manner, such that the product of Formula (I) is obtained as a radiopharmaceutical composition. Thus, the method is carried out under aseptic manufacture conditions to give the desired sterile, non-pyrogenic radiopharmaceutical product. It is preferred therefore that the key components, especially any parts of the apparatus which come into contact with the product of Formula (I) (e.g. vials and transfer tubing) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise the non-radioactive components in advance, so that the minimum number of manipulations need to be carried out on the radioiodinated radiopharmaceutical product. As a precaution, however, it is preferred to include at least a final sterile filtration step.

The precursors of Formula (IA) or (IB), and other reactants, reagents and solvents are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour. The reaction vessel is suitably chosen from such containers, and preferred embodiments thereof. The reaction vessel is preferably made of a biocompatible plastic (e.g. PEEK).

When the radioiodinated guanidine is used as a pharmaceutical composition, the method of the second or third aspects is preferably carried out using an automated synthesizer apparatus. By the term “automated synthesizer” is meant an automated module based on the principle of unit operations as described by Satyamurthy et al [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical product is desired. They are commercially available from a range of suppliers [Satyamurthy et al, above], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. The automated synthesizer preferably comprises a cassette.

By the term “cassette” is meant a piece of apparatus designed to fit removably and interchangeably onto an automated synthesizer apparatus (as defined below), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (eg. solid phase extraction, SPE). The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes of the present invention are designed to be suitable for radiopharmaceutical manufacture and are therefore manufactured from materials which are of pharmaceutical grade and ideally also are resistant to radiolysis.

Preferred automated synthesizers of the present invention are those comprising a disposable or single use cassette which comprises all the reagents, reaction vessels and apparatus necessary to carry out the preparation of a given batch of radioiodinated radiopharmaceutical. The cassette means that the automated synthesizer has the flexibility to be capable of making a variety of different radioiodine-labelled radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

In a fourth aspect, the present invention provides a radiopharmaceutical composition comprising an effective amount of the radioiodinated guanidine of Formula (I) as defined in the first aspect, together with a biocompatible carrier medium.

Preferred embodiments of the radioiodinated guanidine of Formula (I) in the fourth aspect are as defined in the first aspect.

The “biocompatible carrier medium” comprises one or more pharmaceutically acceptable adjuvants, excipients or diluents. It is preferably a fluid, especially a liquid, in which the radioiodinated guanidine of Formula (I) is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The radiopharmaceutical composition of the fourth aspect is suitably sterile. Methods of obtaining such sterile compositions, or of sterilising previously non-sterile compositions are as described in the third aspect (above).

In a fifth aspect, the present invention provides a precursor of Formula (IA) or (IB), as described in the second and third aspects respectively.

Preferred aspects of the Formula (IA) and (IB) in the precursor of the fifth aspect, are as described in the second and third aspects respectively. Preferably, the precursor of the fifth aspect is of Formula (IA).

In a sixth aspect, the present invention provides the use of the precursor of Formula (IA) as defined in the second aspect, or the precursor of Formula (IB) as defined in the third aspect in the manufacture of the radioiodinated guanidine of Formula (I) as defined in the first aspect, or for the manufacture of the radiopharmaceutical composition of the fourth aspect.

Preferred embodiments of the radioiodinated guanidine of Formula (I), precursor of Formula (IA) or of Formula (IB) in the use of the fifth aspect, are as defined in the first, second and third aspects respectively.

In a seventh aspect, the present invention provides the use of an automated synthesizer apparatus to carry out the method of preparation of the second or third aspects.

Preferred embodiments of the precursors, methods and automated synthesizer in the use of the sixth aspect are as described in the second and third aspects.

In an eighth aspect, the present invention provides a method of generating an image of a human or animal body comprising administering the radioiodinated guanidine of Formula (I) of the first aspect, or the radiopharmaceutical composition of the fourth aspect, and generating an image of at least a part of said body to which said compound or composition has distributed using PET or SPECT.

Preferred aspects of the radioiodinated guanidine and radiopharmaceutical composition in the eighth aspect are as described in the first and fourth aspects respectively.

The radioiodinated guanidines of the invention are useful for imaging to assist in the diagnosis of various pathophysiological conditions of the heart, as well as tumour imaging especially of neuroendocrine tumours.

In a further aspect, the present invention provides a method of monitoring the effect of treatment of a human or animal body with a drug, said method comprising administering to said body the radioiodinated guanidine of Formula (I) as defined in the first aspect, or the radiopharmaceutical composition of the fourth aspect, and detecting the uptake of said guanidine or composition in at least a part of said body to which said guanidine composition has distributed using PET or SPECT, said administration and detection optionally but preferably being effected before, during and after treatment with said drug.

The administration and detection of this final aspect are preferably effected before and after treatment with said drug, so that the effect of the drug treatment on the human or animal patient can be determined. Where the drug treatment involves a course of therapy, the imaging can also be carried out during the treatment.

The diseases or conditions being treated in the further aspect are as described in the eighth aspect (above)

The invention is illustrated by the following Examples. Example 1 provides the synthesis of ¹²³I-iodoacetylene. Example 2 provides the click cycloaddition of ¹²³I-iodoacetylene to an azide derivative, to form a radioiodinated triazole ring. Example 3 provides the click cycloaddition of ¹²³I-iodoacetylene to an isonitrile oxide derivative, to form a radioiodinated isoxazole ring. Example 4 provides a click cycloaddition of a tributyltin-alkyne to an azide derivative, to form a triazole radioiodination precursor having a triazole-tributyltin bond. Example 5 provides the conditions for converting the precursor of Example 4, to the radioiodinated product. Example 6 provides a synthesis of an isoxazole radioiodination precursor having an isoxazole-tributyltin bond via click cycloaddition from an isonitrile oxide derivative. Example 7 provides the radioiodination of the precursor of Example 6. Example 8 provides the synthesis of an azidoethyl guanidine. Example 9 provides the synthesis of a triazole-substituted guanidine, having a tributyltin functional group. Example 10 provides the radioiodination of the precursor of Example 9. Example 11 provides the synthesis of an aldoxime-functionalised guanidine. Example 12 provides the synthesis of an isoxazole-substituted guanidine, having a tributyltin functional group. Example 13 provides the radioiodination of the precursor of Example 12.

Abbreviations Used in the Examples.

HPLC: high performance liquid chromatography,

PAA: peracetic acid,

RCP: radiochemical purity,

THF: tetrahydrofuran.

### Example 1

### Preparation and Distillation of [¹²³I]-Iodoacetylene Using Peracetic Acid Oxidant

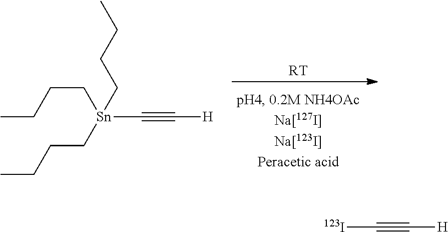

To a Wheaton vial on ice was added, ammonium acetate buffer (100 μl, 0.2M, pH 4), sodium [¹²⁷I] iodide (10 μl, 10 mM solution in 0.01M sodium hydroxide, 1×10⁻⁷ moles), sodium [¹²³I] iodide (20 μl, 53 MBq), peracetic acid, (10 μl, 10 mM solution, 1×10⁻⁷ moles) and a solution of ethynyltributylstannane in THF (Sigma-Aldrich; 38 μl, 1 mg/ml, 1.2×10⁻⁷ moles). Finally, 460 μl THF was added, the Wheaton vial sealed and the reaction mixture allowed to warm to room temperature prior to reverse phase HPLC analysis which showed [¹²³I]-iodoacetylene with a radiochemical purity (RCP) of 75% (t_{R} 12.3 minutes, System A).

The reaction mixture was heated at 80-100° C. for 30 minutes during which time, the [¹²³I]-iodoacetylene and THF were distilled through a short tube into a collection vial on ice. After this time, a low flow of nitrogen was passed through the septa of the heated vial to remove any residual liquids from the tube. [¹²³I]-iodoacetylene was collected in 38.6% yield (non decay corrected) with an RCP of 94%. (t_{R} 12.3 minutes, System A).

HPLC System A (A=water; B=acetonitrile).

Column C18 (2) phenonenex Luna, 150×4.6 mm, 5 micron

Time (min)

| | | 0 | 1 | 20 | 25 | 25.5 | 30 |
|---|---|---|---|---|---|---|---|
| Gradient | % B | 5 | 5 | 95 | 95 | 5 | 5 |

### Example 2

### Preparation of 1-Benzene-4-[¹²³I]-iodo-1H-1,2,3 triazole (Prophetic Example)

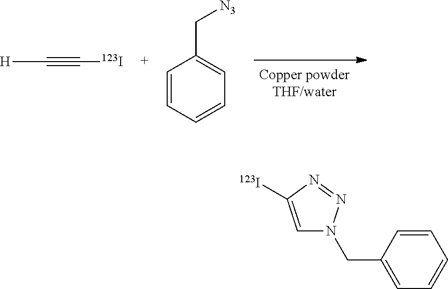

To a Wheaton vial charged with copper powder (200 mg, −40 mesh), sodium phosphate buffer (200 μL, pH 6, 50 mM) and placed on ice is added, [¹²³I]-iodoacetylene and benzyl azide (1 mg, 7.5×10⁻⁶ moles). Following reagent addition, the ice bath is removed and the reaction incubated at room temperature with heating applied as required. 1-Benzene-4-[¹²³I]-iodo-1H-1,2,3-triazole is purified by reverse phase HPLC.

### Example 3

### Preparation of 5-[¹²³I]-Iodo-3-phenyl isoxazole (Prophetic Example)

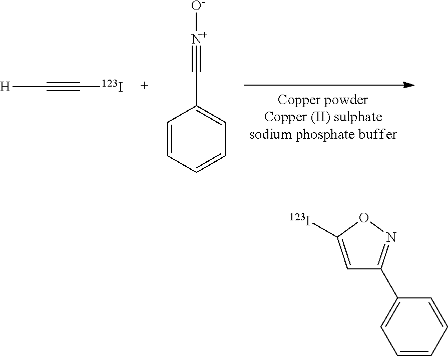

To a Wheaton vial charged with copper powder (50 mg, −40 mesh), copper (II) sulphate (3.8 μg, 1.53×10-8 moles, 0.5 mg/mL solution in water), sodium phosphate buffer (100 μL, 50 mM, pH 6) and placed on ice, is added [¹²³I]-iodoacetylene and benzonitrile-N-oxide (1 mg, 8.4×10⁻⁶ moles. Following reagent addition, the ice bath is removed and the reaction incubated at room temperature with heating applied as required. 5-[¹²³I]-iodo-3-phenyl isoxazole is purified by reverse phase HPLC.

### Example 4

### Preparation of 1-Phenyl-4-(tributylstannyl)-1H[1,2,3]triazole (Prophetic Example)

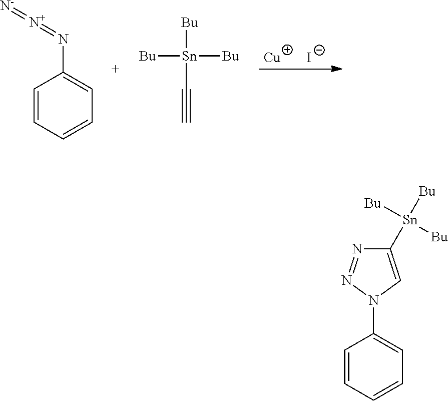

Phenylazide can be obtained from Sigma-Aldrich or can be synthesized by the method described in J. Biochem., 179, 397-405 (1979). A solution of tributylethynyl stannane (Sigma Aldrich; 400 mg, 1.27 mmol) in THF (4 ml) is treated with phenylazide (169 mg, 1.27 mmol), copper (I) iodide (90 mg, 0.47 mmol), and triethylamine (256 mg, 2.54 mmol) at room temperature over 48 h. The reaction is then filtered through celite to remove copper (I) iodide and chromatographed on silica in a gradient of 5-20% ethyl acetate in petrol. The second fraction is collected and concentrated in vacuo to give the 1-phenyl-4-(tributylstannyl)-1H[1,2,3]triazole as a colourless oil.

### Example 5

### Preparation of [¹²³]I-1-phenyl-4-iodo-1H[1,2,3]triazole Using Peracetic Acid as the Oxidant (Prophetic Example)

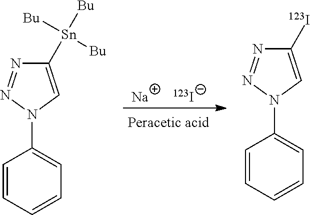

To sodium [¹²³I] iodide, received in 5-20 μL 0.05M sodium hydroxide is added ammonium acetate buffer (100 μL pH 4.0, 0.2M), sodium [¹²⁷I] iodide (10 μL 1 mM solution in 0.01M sodium hydroxide, 1×10⁻⁸ moles), peracetic acid (PAA) solution (10 μL 1 mM solution, 1×10⁻⁸ moles) and finally, 1 phenyl-4-tributylstannyl-1H[1,2,3]triazole (Example 4; 43 μg, 1×10⁻⁷ moles) dissolved in acetonitrile. The reaction mixture is incubated at room temperature for 15 minutes prior to purification by HPLC.

### Example 6

### Preparation of 3-Phenyl-5-(tributylstannyl)isoxazole (Prophetic Example)

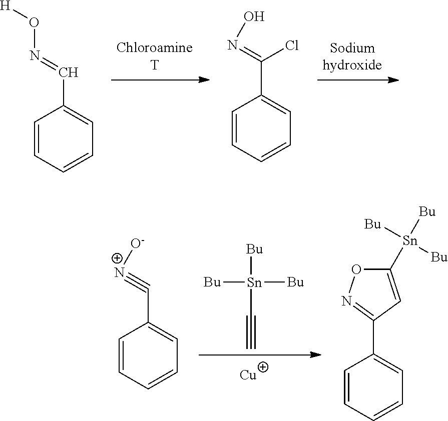

(E)-benzaldehyde oxime (Sigma Aldrich; 3.3 g, 20 mmol) in tert butanol and water (1:1) 80 ml, is treated with chloramine T trihydrate (Sigma Aldrich; 5.9 g, 21 mmol) in small, portions over 5 min. The reaction is then treated with copper sulfate pentahydrate (0.15 g, 0.6 mmol) and copper turnings ˜50 mg and tributylethynylstannane (6.3 g, 20 mmol). The reaction is then adjusted to pH 6 with sodium hydroxide solution and stirred for 6 h. The reaction mixture is treated with dilute ammonium hydroxide solution to remove all copper salts. The product is collected by filtration, redissolved in ethyl acetate and filtered through a short plug of silica gel. The filtrate is concentrated in vacuo to give 3-phenyl-5-(tributylstannyl)isoxazole.

### Example 7

### Preparation of 5-[¹²³I]-iodo-3-phenyl isoxazole (Prophetic Example)

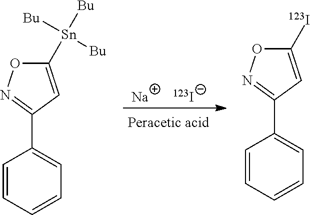

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10⁻⁸ moles), peracetic acid (PAA) solution (10 μL 1 mM solution, 1×10⁻⁸ moles) and finally, 3-phenyl-5-tributylstannyl-isoxazole (Example 6; 43 μg, 1×10⁻⁷ moles) dissolved in acetonitrile. The reaction mixture is incubated at room temperature for 15 minutes prior to purification by HPLC.

### Example 8

### Preparation of N-(2-Azido-ethyl)-guanidine (Prophetic Example)

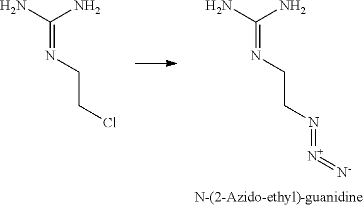 [SMILES: NC(N)=NCCCl ; [N-]=[N+]=NCCN=C(N)N]

N-(2-Azido-ethyl)-guanidine CH19590528 is treated with sodium azide in methanol for 2 h at room temperature. The product is used directly in the next step.

### Example 9

### Preparation of N-(2-(4-tributylstannyl-[1,2,3]triazol-1-yl)-ethyl]-guanidine (Prophetic Example)

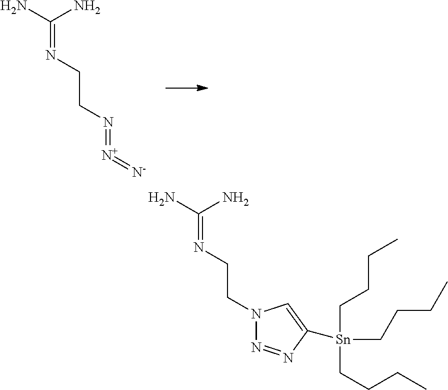

N-(2-Azido-ethyl)-guanidine is reacted with tributylethynylstannane in THF in the presence of a copper (I) iodide catalyst. The reaction is stirred at room temperature for 12 h and the product recovered from the reaction mixture by partitioning between ethyl acetate and water. The ethyl acetate solution is then separated, dried over sodium sulfate and concentrated in vacuo to a gum.

### Example 10

### Preparation of N-(2-(4-Iodo-[1,2,3]triazol-1-yl)-ethyl]-guanidine (Prophetic Example)

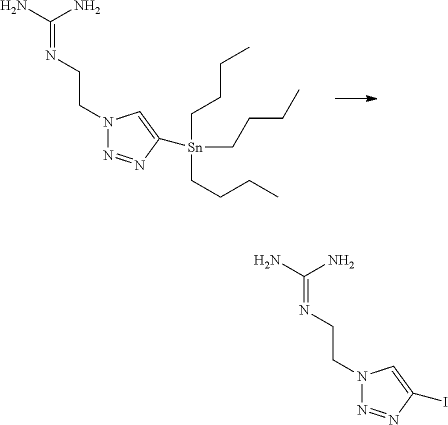 [SMILES: CCC[CH2][Sn]([CH2]CCC)([CH2]CCC)[c]1cn(CCN=C(N)N)nn1 ; NC(N)=NCCn1cc(I)nn1]

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10⁻⁸ moles), peracetic acid (PAA) solution (10 μL 1 mM solution, 1×10⁻⁸ moles) and finally N-(2-(4-tributylstannyl-[1,2,3]triazol-1-yl)-ethyl]-guanidine solution in ethanol or acetonitrile (1×10⁻⁷ moles). The reaction mixture is allowed to stand at room temperature for 15 minutes prior to HPLC purification of the iodinated product.

### Example 11

### Preparation of N-(3-Hydroxyimino-propyl)guanidine (prophetic Example)

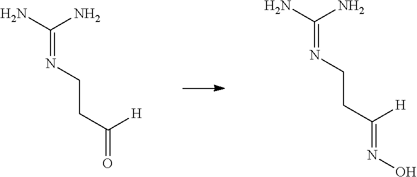 [SMILES: NC(N)=NCCC=O ; NC(N)=NCC/C=N/O]

(3-Ketopropyl) guanidine is dissolved in methanol and treated with hydroxylamine hydrochloride (1 equivalent) and the reaction mixture is allowed to stand at room temperature for 3 h. The reaction is then concentrated in vacuo. The reaction mixture is used directly in the next step.

### Example 12

### Preparation of N-(2-(5-trimethylstannyl-isoxazol-3-yl)-ethyl]-guanidine (Prophetic Example)

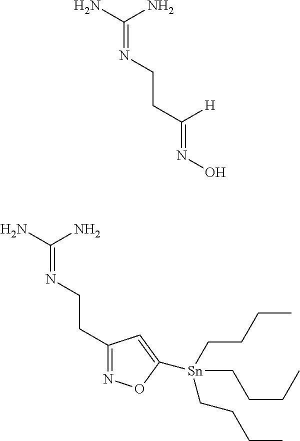 [SMILES: NC(N)=NCC/C=N/O ; CCC[CH2][Sn]([CH2]CCC)([CH2]CCC)[c]1cc(CCN=C(N)N)no1]

N-(3-Hydroxyimino-propyl)guanidine in methanol is treated with chloramine T to oxidise the oxime to a chloro-oxime. The chloro-oxime is then deprotonated by adjusting the reaction mixture to pH 9, and the resulting nitrile oxide reacted with tributylethynylstannane in the presence of a copper (I) iodide catalyst.

### Example 13

### Preparation of N,N-(2-(5-Iodoisoxazol-3-yl)-ethyl]-guanidine (Prophetic Example)

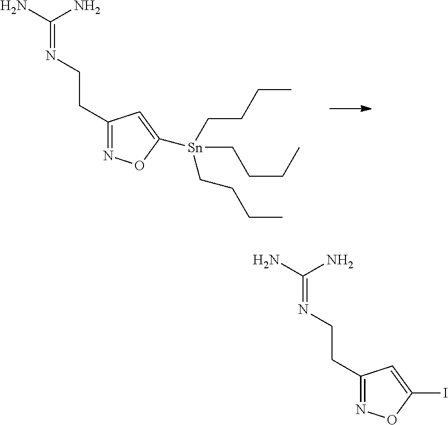

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10⁻⁸ moles), peracetic acid (PAA) solution (10 μL 1 mM solution, 1×10⁻⁸ moles) and finally N-(2-(5-trimethylstannyl-isoxazol-3-yl)-ethyl]-guanidine solution in ethanol or acetonitrile (1×10⁻⁷ moles). The reaction mixture is allowed to stand at room temperature for 15 minutes prior to HPLC purification of the iodinated product.

What is claimed is:

1. A radioiodinated guanidine of Formula (I): 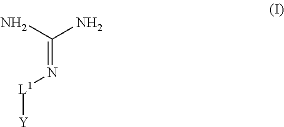
where:
Y is a Y¹ or Y² group: 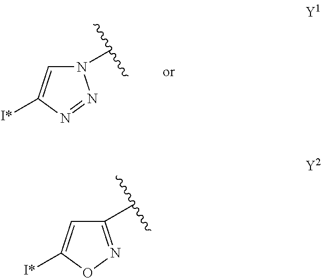
L¹ is a linker group of formula -(A)ₙ- where n is an integer of value 1 to 4, and each A group is independently chosen from —CH₂— and —C₆H₄—;
I* is a radioisotope of iodine.

2. The radioiodinated guanidine of claim 1, wherein I* is chosen from ¹²³I, ¹²⁴I or ¹³¹I.

3. The radioiodinated guanidine of claim 1, where Y is Y¹.

4. The radioiodinated guanidine of claim 1, where L¹-Y is chosen from —CH₂—CH₂—Y and —CH₂—C₆H₄—Y.

5. A method of preparation of the radioiodinated guanidine of Formula (I) as defined in claim 1, where said method comprises:
(i) provision of a precursor of Formula (IA) 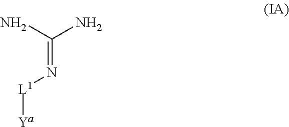
where:
L¹ is as defined in claim 1;
Y^{a} is a Y^{1a} or Y^{2a} group: 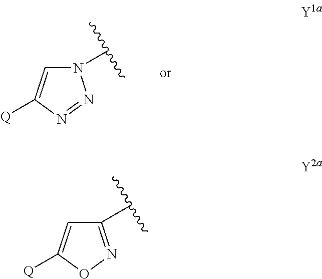
wherein Q is R^{a}₃Sn— or KF₃B—, where each R^{a} is independently C₁₋₄ alkyl;
(ii) reaction of said precursor with radioactive iodide ion in the presence of an oxidising agent to give the radioiodinated guanidine of Formula (I).

6. A method of preparation of the radioiodinated guanidine of Formula (I) as defined in claim 1, where said method comprises:
(i) provision of a precursor of Formula (IB) 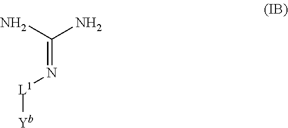
where:
L¹ is as defined in claim 1;
Y^{b} is a Y^{1b} or Y^{2b} group: 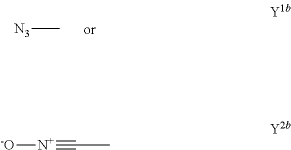
(ii) reaction of said precursor with a compound of Formula (II): 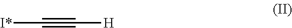
in the presence of a click cycloaddition catalyst, to give the radioiodinated guanidine of Formula (I) via click cycloaddition,
wherein I* is a radioisotope of iodine, as defined in claim 1.

7. The method of claim 6, where the click cycloaddition catalyst comprises Cu(I).

8. The method of claim 6, where the compound of Formula (II) is generated in situ by deprotection of a compound of Formula (IIa): 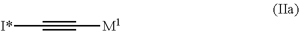
wherein M¹ is an alkyne-protecting group.

9. The method of claim 5, which is carried out in an aseptic manner, such that the guanidine product of Formula (I) is obtained as a radiopharmaceutical composition.

10. The method of claim 5, which is carried out using an automated synthesizer apparatus.

11. A radiopharmaceutical composition comprising an effective amount of the radioiodinated guanidine of Formula (I) as defined in claim 1, together with a biocompatible carrier medium.

12. A precursor of Formula (IA) as defined in claim 5, or of Formula (IB).

13. (canceled)

14. (canceled)

15. A method of generating an image of a human or animal body comprising administering the radioiodinated guanidine of Formula (I) as defined in claim 1, or a radiopharmaceutical composition comprising an effective amount of the radioiodinated guanidine of Formula (I) together with a biocompatible carrier medium, and generating an image of at least a part of said body to which said compound or composition has distributed using PET or SPECT.

16. A method of monitoring the effect of treatment of a human or animal body with a drug, said method comprising administering to said body the radioiodinated guanidine of Formula (I) as defined in claim 1, or a radiopharmaceutical composition of comprising an effective amount of the radioiodinated guanidine of Formula (I) together with a biocompatible carrier medium, and detecting the uptake of said guanidine or composition in at least a part of said body to which said guanidine or composition has distributed using PET or SPECT, said administration and detection optionally but preferably being effected before, during and after treatment with said drug.

17. The method of claim 6, which is carried out in an aseptic manner, such that the guanidine product of Formula (I) is obtained as a radiopharmaceutical composition.

18. The method of claim 6, which is carried out using an automated synthesizer apparatus.

19. A precursor of Formula (IB) as defined in claim 6.
